(19) United States
(12) Patent Application Publication
(10) Pub. No.: US 2006/0099589 A1
(43) Pub. Date: May 11, 2006

(54) Gene shuffling by template switching

(75) Inventors: Henrik Pedersen, Bagsvaerd (DK); Thomas Thisted, Frederikssund (DK); Hans Thorleif Moller, Odense C (DK)

Correspondence Address: BROWDY AND NEIMARK, P.L.L.C.;624 NINTH STREET, NW, SUITE 300, WASHINGTON, DC, 20001-5303, US

(73) Assignee: Nuevolution A/S, Copenhagen (DK)

(21) Appl. No.: 10/522,171

(22) PCT Filed: Jul. 11, 2003
(86) PCT No.: PCT/DK03/00490

§ 371 Date: Nov. 4, 2005

Related U.S. Application Data
(60) Provisional application No. 60/399,156, filed on Jul. 30, 2002.

(30) Foreign Application Priority Data
Jul. 23, 2002 (DK) PA 2002 01130

(51) Int. Cl. C12Q 1/68; C12P 19/34

(52) U.S. Cl. 435/6; 435/91.2

(57) ABSTRACT

The present invention provides a new approach to creating novel polynucleotide sequences by point mutation and recombination in vitro of a set of parental sequences. The new polynucleotide sequences can be useful in themselves or they can be used for the templated synthesis of polymers, e.g. polypeptides composed of α-amino acids as occurring naturally in protein synthesis or polymers comprised of β-amino acids or other building blocks as described (Templated molecules).

### FIELD OF THE INVENTION

The present invention relates to creating novel nucleic acid sequences by recombination in vitro of a set of parental sequences. The parental sequences may for example be prepared by mutagenesis. The novel nucleic acid sequences may be useful per se or they may be utilised as templates for synthesis of polymers, e.g. polypeptides composed of α-amino acids or polymers comprised of β-amino acids or other building blocks.

### BACKGROUND OF THE INVENTION

Protein-engineering technology increasingly draws use of fundamental principles in natural biological evolution. The key processes in evolution are generation of a variety of compounds and selection of favourable variants/features. Through genetic inheritance, selected traits are passed on to progenitors and again subjected to generation of variety. At the heart of evolution lies generation of mutations and recombination events that are reflected in the activity of particular gene products conferring specific traits to an organism. It has now been widely recognized that the same processes can be mimicked in a test tube at the molecular level.

In one version of in vitro evolution, variation is generated at the onset of the experiment and subsequently the desired molecules are selected through an iterative method of selection and amplification. For example, a randomized pool of nucleic acids are selected on basis of binding to a particular ligand and subsequently amplified by the polymerase chain reaction. The same method may be applied for selection of proteins provided that a physical link between the coding nucleic acid and the encoded protein exist. One method of linking the coding nucleic acid and its gene-product is by phage display, where the encoded protein is displayed on a phage that carries the coding information. Also methods for directly linking protein and its encoding mRNA have been developed (Fitzgerald, K., Drug Discov Today June 2000; 5(6):253-258).

Instead of or in addition to generating variation at the onset of the experiment, variation may also be generated during the course of the experiment by employing mutagenic PCR.

Another in vitro evolution method that has proven extremely successful for protein engineering is the method of gene shuffling. In this method, normally the starting material is DNA sequences encoding homologues proteins. The starting material may be homologues genes from different organisms or a mutated pool of one particular gene. Gene shuffling is the process of recombining the starting pool of sequences to generate new gene-sequences that subsequently can be screened for particular desired characteristics.

An artificial version of sexual reproduction was invented by Stemmer (U.S. Pat. No. 6,297,053). This method relies on homologous recombination during the PCR assembly of random gene fragments from multiple homologous parents, and generates crossovers in regions of relatively high-level DNA homology and at loci of sequence identity. Random cleavage of double-stranded DNA corresponding to the homologous parental sequences is most commonly achieved by partial DNase I digestion, but also fragmentation by uracil-DNA-glycosylase after incorporation of uracil in the parental DNA has been claimed. The fragmented DNA is subsequently used in a PCR reaction without external primers during which homologous sequences will anneal and prime the synthesis of recombined DNA. To amplify full length DNA sequences, primers complementary to the 5′- and 3′-ends are added in the last PCR cycles.

A similar approach has been introduced by Arnold et al. (U.S. Pat. No. 6,153,410). Their StEP (staggered-extension) protocol utilizes PCR with repeated cycles of denaturation and extremely short annealing/polymerase catalyzed extension times. Thus, short incompletely extended products are generated starting from primers annealing to the 5′- and 3′-ends of the parental genes. In each cycle the growing fragments can anneal to different templates based on sequence complementarity and extend further. The process is repeated until full-length gene is formed. Other similar formats employing synthesis interruption (“stuttering”) using chemicals or radiation has been claimed (U.S. Pat. No. 5,965,408).

Similarly, the protocol termed “Random priming in vitro recombination” (Shao et al., (1998), Nucleic Acids Res., 26, 681-683) involves priming double-stranded template polynucleotides with excess random-sequence primers and extending with polymerase to generate a pool of short DNA fragments. The short DNA fragments can prime on one another based on sequence homology and be recombined and reassembled into full-length genes by repeated rounds of PCR.

The above mentioned shuffling formats all depend on a relatively high level of homology as the fragmented parental sequences would otherwise not hybridize efficiently to each other during the assembly step. The Rachitt protocol (random chimeragenesis on transient templates; Coco et al., (2001), Nature Biotechnology, 19, 354-359) somewhat alleviates this problem reporting the shuffling of low homology parental gene sequences. Rachitt relies on the ordering, trimming and joining (by utilizing nucleases, gap fill-in and ligation) of randomly cleaved parental single-stranded DNA fragments of the same polarity annealed onto a transient complementary polynucleotide template. This template is constituted by a full-length single-stranded homologous gene (differing from any of the fragmented parental genes), which serves as a scaffold for the process. The scaffold (carrying incorporated uracil nucleotides) is rendered non-amplifyable by uracil-DNA-glycosylase treatment, and the shuffled fragments are subsequently amplified by PCR.

A further in vitro technique for shuffling heterologous sequences has been developed by Borchert et al., 2001 (U.S. Pat. No. 6,291,165). At least one conserved sequence region is identified wherein a high degree of homology between the (partially) heterologous sequences exists. The conserved region(s) are then utilized as homologous linking points for shuffling/recombining the parental sequences by SOE-PCR (sequence overlap extension PCR). This method yields only limited diversity in the resulting library, since the crossover point(s) are pre-defined by the experimental setup (i.e. PCR primer design.

Two shuffling methods for sequences with no homology has been reported. Itchy (Iterative truncation for the creation of hybrid enzymes) provides non-homologous recombination based on generation of N— and C-terminal fragment libraries of two genes by progressive truncation of the coding sequences with Exonuclease III followed by ligation of the products to make a “head-to-tail” single-crossover hybrid library (Ostermeier et al., (1999), Nature Biotechnology 17, 1205-1209). The draw-back of the Itchy protocol (library members containing only one crossover per gene) has been alleviated by the introduction of a method termed “Scratchy. By first performing the Itchy protocol followed by standard DNA shuffling, multiple crossover DNA libraries independent of sequence identity can be created (Lutz et al., (2001), Proc. Natl. Acad Sci. 98, 11248-11253).

Additional recent reviews on the subject are: Kurtzman et al., Curr Opin Biotechnol 2001, 12, 361-370, Coco et al., Nat Biotechnol, 2001, 19, 354-359 and Kolkman and Stemmer, Nat Biotechnol, 2001, 19, 423-428.

A key process in the lifecycle of human immunodeficiency virus (HIV) virus is the conversion of the single-stranded RNA genome into a double-stranded DNA genome. This process called reverse transcription is catalyzed by the virally encoded protein; reverse transcriptase. During reverse transcription, inter or intramolecular template switches may take place, a phenomenon known as copy-choice, template switching or strand transfer. Intramolecular strand transfer reactions are responsible for the generation of insertions and deletions, whereas intermolecular template switches eventually yield homologues recombination (Negroni and Buc, 2000, PNAS, 97, 6385-6390).

### SUMMARY OF THE INVENTION

Prior art methods for gene shuffling involve hybridisation of product to template. Accordingly, templates must be sufficiently long to allow specific hybridisation.

The present invention provides novel methods of gene shuffling. The methods utilise template switching during template dependent nucleotide polymerisation to generate novel sequences. The methods are not dependent on the length of templates and accordingly even very short templates may be shuffled according to the invention.

In contrast to prior art shuffling methods, the individual input polynucleotide sequences (templates) exploited in the present invention are all single-stranded and of the same polarity. The decoding of all of the nucleotide templates thus occurs in the same orientation.

Accordingly, template switched products result from polymerase directed, uni-directional decoding of a plurality of templates, such as two templates, or more than two templates.

Without being bound by theory, template switched products are obtained according to one presently preferred hypothesis by decoding at least two separate nucleotide templates, thereby providing a single, continuous template switched product. The template switched product is obtained in one embodiment by firstly decoding at least part of a first template (in the 3′-5′ orientation with respect to the first template), and subsequently decoding at least part af at least one second template having the same polarity (i.e. 3′-5′ orientation with respect to the at least one second template), wherein said decoding process results in the formation of a single, continuously growing template switched product, essentially without giving rise to any other products, such as intermediate products capable of hybridising or annealing to one another.

The templates according to the invention are single stranded and all of the templates have the same polarity and are thus all capable of being decoded in the same direction. A short oligonucleotide primer (typically 15-20 nucleotides) complementary to sequences in the 3′-end of at least one template can be used for initiation of enzymatic nucleotide polymerization (e.g. reverse transcription). Alternatively, the at least one template can contain a hair-pin loop-like structure in the 3′-end resulting from part of the template folding back on itself. Such a structure can also be used for priming. However, without being bound be theory, nucleotide polymerization does not necessarily require any of the aforementioned alternatives.

It is a first objective of the present invention to provide methods of preparing a template switched product, or a plurality of different template switched products, wherein said product(s) is encoded by at least part of one first template and at least part of at least one second template, wherein said template switched product comprises at least one predetermined property, said method comprising the steps of
i) providing one first and at least one second template molecule; and
ii) providing a nucleic acid polymerase; and
iii) synthesising a plurality of different template switched products by contacting sequentially in any order, or simultaneously, at least part of the first template and at least part of the at least one second template with said polymerase under conditions allowing for template dependent nucleotide polymerisation,
   wherein the synthesis of each individual template switched product involves at least one template switch,
   and wherein the synthesis of the plurality of different template switched products involves a plurality of template switches,
iv) separating at least one template switched product comprising the at least one predetermined property from said plurality of template switched products; and
v) obtaining a template switched product comprising at least one predetermined property

Different template switched products are obtained when a polymerase is switching from one location on a first template to different locations on a second template. The template switched product will thus contain smaller or larger parts of the first and second template, respectively, depending on the exact position/location where the template switch occurred.

The template switched product can contain either a part of the first template, or the entire first template (in which case the template switching has occurred at the very end of the first template), as well as part of the second template, or the entire second template (for the same reason as stated above). However, template switching involving a first template and a second template can also produce template switched products comprising multiple fragments in any order of said first and/or second template, but retaining the same polarity/orientation as in the first and/or second template. Such template switched products will result from e.g. more than one template switch between a first and a second template. In one such example the polymerase starts by decoding a first template, switches to a second template, and switches back to a first template. As more copies of the first and second template can be present, the polymerase does not need to switch back to the same identical template which was initially decoded. Switching to another copy of the first template will also result in a continuous, template switched product comprising (in this order) part of the first template linked to part of the second template linked to part of the first template.

As e.g. “the first template” as used herein above merely indicate any one of a number of identical copies of first templates, template switching between a first template and at least one second template shall be understood to include template switching among any copy of such templates present in the reaction mixture. This is to say that template switching during template switched product synthesis can occur once between the same two template copies, or more than once between the same two template copies. Irrespective of which of the aforementioned two alternatives take place (one or more template switches between the same two templates), template switched product synthesis can additionally involve template switches also involving the same or different third and fourth templates, wherein one or more template switches can occur between said third and fourth templates.

In summary, different template switched products according to the present invention can be generated by template switching involving two templates and at least one template switch, such as two template switches involving the same templates (identical templates, or identical copies of said templates), for example three template switches, Alternatively or in addition to the aforementioned, different template switched products according to the present invention can be generated by template switching involving more than two templates, wherein all of said templates are involved in at least one template switch, i.e. the polymerase either starts decoding said template, or switches to said template during template switched product synthesis.

It is an objective of the present invention to provide uses of the above-mentioned methods for gene shuffling.

In one embodiment of the present invention the nucleic acid polymerase is a reverse transcriptase derived from HIV-1 (human immunodeficiency virus-1). It has surpringsingly been demonstrated by the inventors that the inherent feature of HIV-1 reverse transcriptase for recombining homologues sequences (Peliska and Benkovic, Biochemistry, 1994, 33, 3890-3895; Peliska and Benkovic, Science, 1992, 258, 1112-1118), may be employed for gene shuffling.

### BRIEF DESCRIPTION OF THE FIGURES

FIG. 1 illustrates one example of gene shuffling by means of template switched product synthesis. Target sequences can be PCR amplified and this creates DNA templates for RNA synthesis in vitro. Naturally occuring polymerases or polymerases modified by recombinant gene technology or protein engineering can be used, including Reverse Transcriptase and T7 RNA polymerase, as well as other template switching polymerases. When it is desirable to facilitate fragmentation with iodine, phosphorothioate ribonucleotide analogues can be incorporated. After fragmentation, the sequences to be shuffled can be mixed, subjected to template switching, or recombined. Variations in sequence for the two sequences are indicated by letters in upper and lower case, respectively.

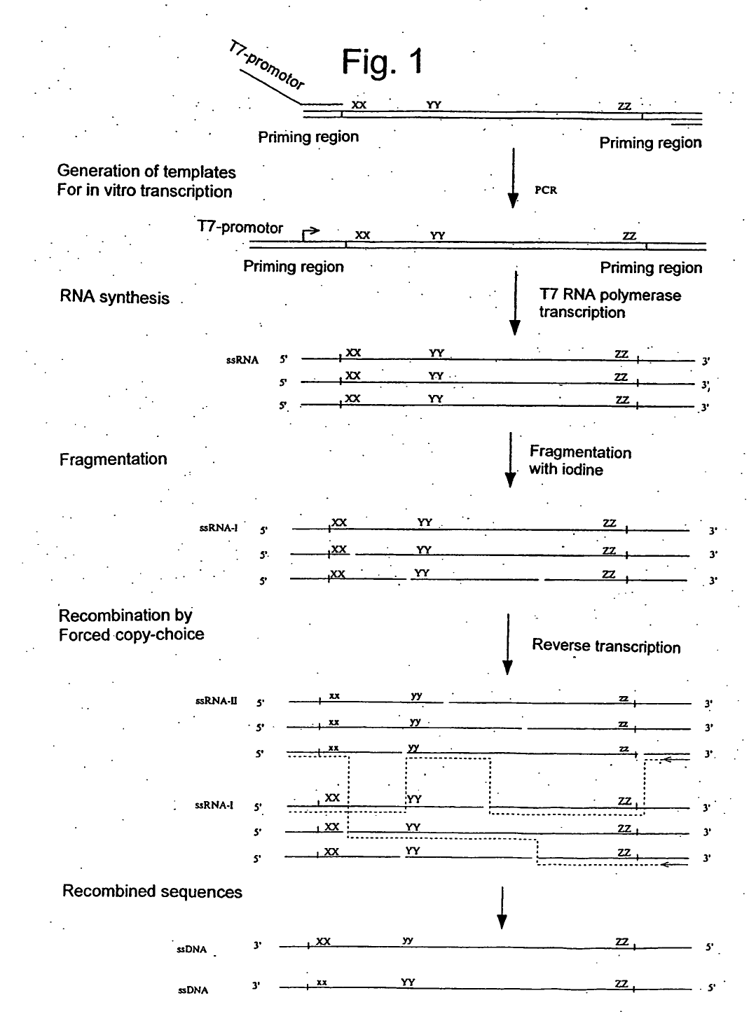

FIG. 2 illustrates principal differences between conventional shuffling methods (illustrated by the shuffling protocol in panel A) and the present invention (panel B).

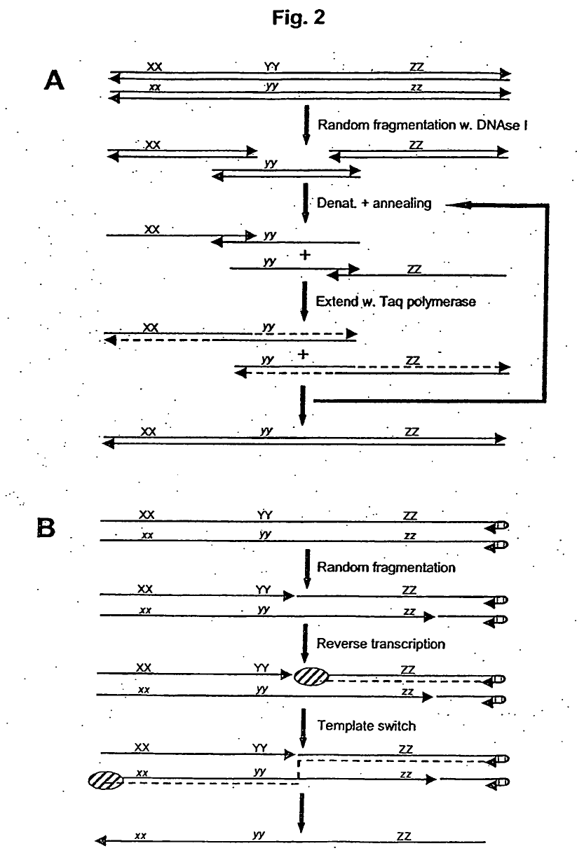

(A) In conventional shuffling protocols, sequences of both polarities (in this case double-stranded DNA) are used for input material. The arrow pointing back to the denaturation and annealing step indicates the cyclic nature of the process where repeated cycles of denaturation, annealing to new complementary strands and extension w. Taq polymerase is required for PCR based assembly of the gene fragments into the full-length product.

(B) In the present invention all input sequences are single stranded and of the same polarity. In this example, random fragmentation of the input RNA causes template switching by reverse transcriptase resulting in the synthesis of the template switched product carrying a recombination of the sequences present in the two input templates. In the present example, reverse transcription is initiated from a short hairpin structure in the 3′-end of the templates. Reverse transcription can also be initiated from a short (typically 15-20 nt) DNA oligonucleotide annealed to the very 3′-end of the templates.

The polarity of the polynucleotide templates (5′ to 3′ direction) is indicated by arrow-heads. Variation in sequence between the parental input sequences are indicated by letters in upper and lower case, respectively. Reverse transcriptase is indicated by a cross-hatched oval.

### DEFINITIONS

Activity

The term “activity” refers to any specific activity that a molecule is capable of performing or encode. For example activity may be that a molecule is capable of associating with a specific binding partner with a specific affinity, capable of catalysing a specific reaction, capable of inhibiting a specific reaction, capable of effecting a particular cellular response, capable of interacting with a physical surface.

According to the present invention a first activity is usually associated with a first template or a product encoded by said first template, and a second activity is usually associated with a second template or a product encoded by said second template.

A third activity is usually associated with a template switched product or a product encoded by a template switched product. A third activity is said to be different to a first activity or a second activity when if its different activity can be determined by using any assay capable of demonstrating physical or functional activities associated with any of the aforementioned templates. By way of example, association with a specific binding partner with a higher or lower affinity or specificity is a different activity.

Conditions Allowing for Template Dependent Nucleotide Polymerisation

“Conditions allowing for template dependent nucleotide polymerisation” are any conditions under which a nucleic acid polymerase is capable of catalysing template dependent nucleotide polymerisation.

The specific conditions will depend on the specific nucleic acid polymerase. In general however, conditions allowing for template dependent nucleotide polymerisation comprises nucleotides, buffer and salt(s). Furthermore, the specific conditions may involve incubation at a specific temperature.

Decoding

Decoding is the process whereby the information content of one nucleotide template is transferred by means of a nucleic acid polymerase to another template, or to a biomolecule. See “Encoding”.

Encoding

A nucleic acid or oligonucleotide analog is said to encode another molecule, if the sequence of said nucleic acid or oligonucleotide analog specifies the sequence or composition of said other molecule. Examples of encoding are: i) RNA encodes a DNA when a RNA molecule is reverse transcribed by e.g. a reverse transcriptase to form a DNA strand whose sequence is complementary to the RNA; ii) mRNA encodes a peptide when the genetic code of the mRNA is translated into a peptide by a ribosome, where the sequence of the peptide is determined by the sequence of triplet codons of the mRNA; iii) DNA encodes a polymer or scaffolded molecule, when the single stranded DNA is first replicated using nucleotide and/or nucleotide derivatives and a polymerase, whereafter appendices attached to the nucleotides/nucleotide derivatives are covalently linked to form a polymer or scaffolded molecule, where the sequence or composition of the polymer or scaffolded molecule, respectively, is determined by the sequence of the template DNA strand.

Gene Shuffling

Gene shuffling is the process wherein one or more nucleic acids are produced comprising sequences derived from two or more different starting nucleic acids. Gene shuffling may for example be used to obtain novel nucleic acids with specific activity.

Nucleic Acid Polymerase

A nucleic acid polymerase is a catalyst, e.g. an enzyme or ribozyme, capable of catalysing template dependent polymerisation of nucleotides.

Preferred nucleic acid polymerases according to the invention are reverse transcriptases, i.e. enzymes capable of catalysing polymerisation of deoxynucleotides using a ribonucleic acid as template.

In one preferred aspect of the present invention the reverse transcriptase is derived from a human immunodeficiency virus (HIV).

Nucleotides

Nucleotides according to the invention includes ribonucleotides comprising a nucleobase selected from the group consisting of adenine (A), uracil (U), guanine (G), and cytosine (C), and deoxyribonucleotide comprising a nucleobase selected from the group consisting of adenine (A), thymine (T), guanine (G), and cytosine (C).

Nucleobases are capable of associating specifically with one or more other nucleobases via hydrogen bonds. Thus it is an important feature of a nucleobase that it can only form stable hydrogen bonds with one or a few other nucleobases, but that it can not form stable hydrogen bonds with most other nucleobases usually including itself. The specific interaction of one nucleobase with another nucleobase is generally termed “base-pairing”.

The base pairing results in a specific hybridisation between predetermined and complementary nucleotides. Complementary nucleotides according to the present invention are nucleotides that comprise nucleobases that are capable of base-pairing.

Of the naturally occurring nucleobases adenine (A) pairs with thymine (T) or uracil (U); and guanine (G) pairs with cytosine (C). Accordingly, e.g. a nucleotide comprising A is complementary to a nucleotide comprising either T or U, and a nucleotide comprising G is complementary to a nucleotide comprising C.

Nucleotide Analogues

Nucleotide analogues include any nucleotide analogues capable of specific bas-pairing, for example nucleotide analogues may be derivatives of naturally occurring nucleotides, or they may comprise a different backbone or different base moiety than naturally occurring nucleotides. Nucleotide analogues according to the present invention are capable of specific hybridisation with at least one other nucleotide; and association with the active site of at least one nucleic acid polymerase.

Examples of nucleotide analogues according to the invention includes but are not limited to PNA, LNA, dideoxynucleotides, deoxyribonucleotides, and nucleotides containing unnatural bases.

Predetermined Property

The predetermined property of a template switched product may be any desirable property. For example the predetermined property may be specific association with a specific binding partner or the property may be that the template switched product encodes another product with a predetermined activity.

Scaffolded Molecule

A scaffolded molecule according to the present invention is a molecule comprising a scaffold to which more than 2 chemical entities are attached. Said chemical entities may for example be monomer units capable of constituting part of a polymer.

A scaffold within the meaning of the present invention is a molecular moiety to which more than 2 chemical entities may be directly attached. Hence a scaffold preferably comprises more than two reactive groups.

Template

The template is preferably a single strand of nucleotides or nucleotide analogs. When the template comprises a strand of nucleotides, the nucleotides may be natural or non-natural, and may be linked by e.g. phosphorothioate bonds or natural phosphodiester bonds. Nucleotide analogs may be linked e.g. by amide bonds, peptide bonds, or any equivalent means capable of linking nucleotide analogs. The sugar moiety of a nucleotide or nucleotide analog may be a ribose or a deoxyribose, a ribose derivative, or any other molecular moiety.

A template comprises a sequence of n coding elements, wherein n is an integer of more than 5. A coding element is an element that directs insertion of a specific group, for example a specific nucleotide or nucleotide analogue into the product.

In one preferred embodiment of the present invention, the template is a nucleic acid or a nucleic acid analogue. More preferably, the template is a nucleic acid, which is capable of being transcribed by a polymerase comprising RNase H activity, including a reverse transcriptase. Accordingly, it is preferred that the starting template comprises or consists of RNA or a derivative or analogue thereof. The methods according to the invention may involve use of more than one template, for example the method may involve the use of a first and a second template.

Template Switch

Template switching occurs when a nucleic acid polymerase which is synthesizing a templated product (template switched product) by decoding at least part of a first template, shifts from said first template to a second template, which is subsequently at least partly decoded during the synthesis of the remaining part of said templated product. It is possible for the polymerase to decode (from the location of the switch) the entire remaining part of the second template, to decode part of said remaining part of said second template and, in the latter case, to switch template again to e.g. any position of said first template (or an identical copy thereof), or switch to any other template.

The templated product is thus synthesized by a combination of nucleic acid polymerase directed decoding of at least part of said first template and nucleic acid polymerase directed decoding of at least part of said second template. Template switching and synthesis of a template switched product according to the invention involves the same nucleic acid polymerase (unlike PCR where different polymerases are used to decode templates having different polarity) and the synthesis of a template switched product is one continuous process that does not involve denaturation and renaturation of double stranded nucleic acids (unlike PCR).

Template Switch Signal

A template switch signal is any modification of a template, capable of inducing a higher rate of template switches of a nucleic acid polymerase. A template switch signal can be introduced into a template simultaneously with synthesis of said template or the template switch signal may be introduced after synthesis of the template. Preferably, the template switch signal is a signal capable of introducing a pause in nucleotide polymerisation catalysed by a nucleic acid polymerase.

Template Switched Product

A template switched product according to the present invention is the product of a process involving contacting sequentially in any order or simultaneously at least part of a first template and at least part of a second template with a nucleic acid polymerase under conditions allowing for template dependent nucleotide polymerisation, wherein the synthesis of each individual template switched product involves at least one template switch. The template switched product is synthesised in a continuous process as the length of the template switched product strand increases during decoding of (initially) the first template, or a part thereof, and (subsequently) during decoding of the second template, or a part thereof. Thus, it is possible to regard the template switched product as a chimera comprising first template sequence (or a one to one relationship with such a sequence), and second template sequence, (or a one to one relationship with such a sequence).

The template switched product is preferably a nucleic acid. Preferably, the template switched product is a nucleic acid, which may be the product of a reverse transcription reaction. Accordingly, it is preferred that the template switched product comprises or consists of DNA, or a derivative or analogue thereof.

### DETAILED DESCRIPTION OF THE INVENTION

Methods of Preparing Template Switched Products

The present invention relates to methods of preparing a template switched product encoded by at least part of one first and part of one second template, wherein said product comprises at least one predetermined property, said method comprising the steps of
i) providing at least one first and at least one second template molecule; and
ii) providing a nucleic acid polymerase; and
iii) synthesising a plurality of different template switched products by contacting sequentially in any order or simultaneously at least part of the first template and at least part of the at least one second template with said polymerase under conditions allowing for template dependent nucleotide polymerisation,
   wherein the synthesis of each individual template switched product involves at least one template switch, and
   wherein the synthesis of the plurality of different template switched products involves a plurality of template switches,
iv) separating at least one template switched product comprising the at least one predetermined property from said plurality of template switched products; and
v) obtaining a template switched product comprising at least one predetermined property

Such methods may be useful for several purposes, however, the methods are in particular useful for gene shuffling. During sexual reproduction homologous recombination contributes to the continous evolution of the genome. Gene shuffling is an vitro process mimicking natural evolution, by creating novel nucleic acid sequences by “shuffling” two or more template nucleic acid sequences. Subsequent to shuffling of the sequences, sequences comprising desired properties can be selected.

The individual steps of the methods may be performed more than once. In particular, it is frequently preferred that the entire sequence of steps are repeated at least once. For example the sequence of steps may be repeated once, such as twice, for example 3 times, such as 4 times, for example 5 times, such as in the range of 5 to 10 times, for example in the range of 10 to 20 times, such as in the range of 20 to 50 times, for example more than 50 times.

In particular, when the method is repeated, it is preferred that the template switched products comprising at least one predetermined property obtained by performed the method the first time are used as templates, such as first templates, second templates and/or further templates when the method is repeated. Hence, template switched product(s) may be used as a first template and/or second template when the method is repeated. Alternatively, it is preferred that the method is repeated using templates encoded by the template switched products comprising predetermined property(ies) obtained by performing the method the first time.

By way of example, if the templates are RNA molecules and the template switched products are DNA molecules, novel templates may be synthesised by transcription of said DNA molecules.

By using the template switched products obtained from the method as templates for subsequent rounds of the method, it is possible to obtain template switched products comprising improved predetermined property(ies).

It should be noted that conditions during any of the steps in the method employing template directed polynucleotide synthesis can be modulated as to decrease the fidelity of polynucleotide synthesis. In some cases the added sequence diversity generated by error-prone conditions can be advantageous. This also allows for sequence evolution during repeated rounds of the method, such that a resulting template switched product can contain sequence information (e.g. mutations) not present in the input templates used at the beginning of the method.

In one preferred embodiment the method furthermore comprises the step of introducing one or more template switch signals into the first template, and/or the second template and/or any further templates. Preferably said step is performed subsequent to step i) and prior to step iii). Examples of suitable template switch signals are given herein below.

Prior art gene shuffling methods may require a heat denaturation/renaturation type of annealing between the template(s) and the generated, recombined product(s). It is one advantage of the present invention, that it is not required that the template switched product(s) anneals in this way (heat denaturation/renaturation type of annealing) to any of the templates.

Hence, in one embodiment the methods described herein can involve annealing between the first template and the template switched product, but said methods do not need to comprise such a feature. Furthermore, preferably the methods do not require and preferably do not involve annealing between the second template and/or any further template and the template switched product.

In one preferred embodiment of the invention the synthesis of template switched products involves a reverse transcription reaction. The templates (first template, second templates and any further templates) for a reverse transcription reaction are RNA molecules or analogues thereof. A reverse transcription reaction may for example be performed as follows:

RNA templates are mixed in appropriate ratios, a reverse primer is annealed to the template RNAs and reverse transcription is initiated by the addition of reverse transcriptase, such as HIV reverse transcriptase. After reverse transcription, which should involve at least one template switch, in general a PCR is performed on the produced ssDNA (=template switched products) to facilitate screening or selection for a desired property of the template switched products or to generate templates for another round of synthesis of template switched products.

Suitable reverse primers includes any primer capable of annealing to the template RNA.

Templates

Templates to be used with the present invention may be any template according to the definition herein above. Preferably the template is a polynucleotide comprising or consisting of nucleic acids and/or nucleic acid analogues.

The methods described herein involve the use of at least a first template and at least one second template. However, the methods can involve the use of more than 2 different templates, for example 3, such as in the range of 3 to 5, for example 5 to 10, such as 10 to 20, for example 20 to 50, such as 50 to 100, for example 100 to 250, such as 250 to 500, for example 500 to 1000, such as more than 1000 different templates.

The methods can involve template switching between 2 different templates, or more than 2 different templates, for example 3, such as in the range of 3 to 5, for example 5 to 10, such as 10 to 20, for example 20 to 50, such as 50 to 100, for example 100 to 250, such as 250 to 500, for example 500 to 1000, such as template switching between more than 1000 different templates.

The method in yet another embodiment method involves more than 2 different template switches, such as 3, for example 4, such as 5, for example in the range from 5 to 10, such as from 10 to 20, for example from 20 to 50, such as from 50 to 100, for example from 100 to 200, such as more than 200 different template switches.

One or more of the template switches of the present method are different or unique switches involving the formation of different or unique template switched products. It is to be understood that such products are the result of at least one switch from a predetermined position of a first template to a different position of at least one second or further template, or the result of at least one switch from a different position of a first template to a predetermined position of at least one second or further template, or the result of at least one switch from a different position of a first template to a different position of at least one second or further template, wherein switches to or from different positions are unique for the method, and wherein the method can involve at least one other switch to or from said predetermined position(s). Accordingly, each different template switched product of the invention is the result of at least one unique template switching event, i.e. a template switching event being different from other template switching event(s), thus generating a unique boundary in the continuous, template switched product between sequence originating from the first template and sequence originating from the at least one second template, or a unique boundary between sequence originating from the at least one second template and any further template.

In one embodiment the number of different template switched products generated is preferably more than 20, for example more than 50, such as more than 500, for example more than 1000, such as more than 10000, for example more than 100000, such as more than 1000000. In another embodiment the number of different template switched products generated is preferably in the range of from 1 to 1000000, such as from 1 to 500000, for example from 5 to 500000, such as from 5 to 250000, for example from 10 to 200000, such as from 10 to 150000, for example from 15 to 100000, such as from 5 to 250000, for example from 10 to 200000, such as from 10 to 150000, for example from 15 to 100000, such as from 20 to 200000, for example from 20 to 150000, such as from 25 to 150000, for example from 25 to 125000, such as from 25 to 100000, for example from 50 to 100000, such as from 50 to 50000, for example from 100 to 50000.

It is thus within the scope of the present invention to provide in a single reaction mixture more than 10 different templates and to obtain more than 100 different template switched products.

In a preferred embodiment of the present invention the first template, the second template and any further template are individually selected from the group consisting of nucleic acid molecules and analogues thereof.

The first template, the second template and any further template may pair-wise be identical, similar or different. Preferably, however the first, the second and any further templates are pair-wise similar to one another, but not identical. For example, the first, the second and any further template may pair-wise individually share in the range of 10 to 99%, such as 20 to 99%, for example 30 to 99%, such as 40 to 99%, for example 50 to 99%, such as 60 to 99%, for example 70 to 99%, such as 80 to 99%, for example 90 to 99%, such as 95 to 99%, for example 30 to 95%, such as 40 to 95%, for example 50 to 95%, such as 60 to 95%, for example 70 to 95%, such as 80 to 95%, such as 90 to 95%, for example 40 to 90%, such as 50 to 90%, for example 60 to 90%, such as 70 to 90%, for example 80 to 90% identity.

Furthermore, it is possible that the first, the second and any further template may pair-wise individually share a region comprising 2, for example 3, such as 4, such as 5, such as 6, for example 7, such as 8, for example 9, such as 10, for example 11, such as 12, for example 13, such as 14, for example 15 nucleotides which share at least 40%, such as at least 50%, for example at least 60%, such as at least 70%, for example at least 80%, such as at least 90%, for example at least 95%, such as at 99%, for example around 100% identity.

Hence, for example a region comprising 5 nucleotides in the first template may share at least 40%, such as at least 60%, for example at least 80%, such as around 100% identity with a region comprising 5 nucleotides in the second template.

Preferably, templates according to the invention are any nucleic acid or nucleic acid analogue capable of being replicated or transcribed by a nucleic acid polymerase. Hence, depending of the nucleic acid polymerase that may be useful for the particular embodiment of the invention, the template should preferably be selected so that it may serve as template for that particular nucleic acid polymerase.

In one preferred embodiment of the invention the nucleic acid polymerase is a reverse transcriptase, more preferably HIV reverse transcriptase. Hence, in that embodiment of the invention it is preferred that the first template, the second template and any other templates are selected from the group consisting of templates that are capable of being template for reverse transcriptase.

Accordingly, in that embodiment it is preferred that the first template and/or the second template and/or any further templates are RNA molecules or RNA analogues.

In one embodiment of the invention the first template and/or the second template and/or any further templates encodes a polymer. Said polymer may be any of the polymers described herein below.

In another embodiment of the invention the first template and/or the second template and/or any further templates encodes a scaffolded molecule. Said scaffolded molecule may be any of the scaffolded molecules described herein below.

Template Switched Product

A template switched product according to the present invention is a product of a method involving contacting at least part of a first template and at least part of a second template with a nucleic acid polymerase under conditions allowing for template dependent nucleotide polymerisation, wherein the synthesis of each individual template switched product involves at least one template switch.

The first and the second template may be contacted by the polymerase simultaneously or sequentially in any order. When performing such a method, usually a plurality of different template switched products are generated, wherein the synthesis of each template switched product involves one or more template switches, which are different to the template switches in the synthesis of different template switched products.

Hence, the template switched products according to the invention are products of a nucleic acid polymerase catalysed template dependent nucleotide polymerisation. Accordingly, one or more template switched products according to the present invention comprises or consists of nucleic acids or nucleic acids analogues. Preferably, all template switched products comprises or consists of nucleic acids or nucleic acids analogues.

In one embodiment of the invention at least one, but preferably all template switched products are nucleic acids.

The present invention also relates to separating at least one template switched product with a predetermined property from the plurality of template switched products synthesised.

Said predetermined property may be an intrinsic activity of the template switched product or the predetermined property may be that the template switched product encodes a molecule comprising a desired activity.

For example, the template switched product may be a nucleic acid or nucleic acid analogue with catalytic properties, for example the template switched product may comprise or consist of a ribozyme. Alternatively, the template switched product may be a nucleic acid or nucleic acid analogue capable of interfering with transcription and/or translation of one or more nucleic acids, for example the template switched product may be an antisense or antigene nucleic acid or nucleic acid analogue.

Frequently, the template switched product according to the present invention encodes another molecule. For example, the template switched product may encode a polymer, such as any of the polymers described herein below. “Encode” should be understood to designate the process in which the sequence of a nucleic acid or nucleic acid analogue is translated or transcribed/translated into a molecule, such as a polypeptide by one or more enzymatically catalysed processes. Hence, for example the template switched product may encode a polypeptide.

It is also comprised within the present invention that the template switched product may encode a molecule, which is not a linear polymer, for example the template switched product may encode a scaffolded molecule, such as any of the scaffolded molecules described herein below.

It is also comprised within the scope of the present invention, that the template switched product may be amplified after synthesis. For example, the methods described herein may comprise amplification of the plurality of template switched products. Said amplication may be performed prior to separating template switched products comprising a specific predetermined property from the plurality of template switched products. However, it is also possible that only the template switched products comprising a specific predetermined property are amplified.

Amplification may be performed by a number of different methods that should be selected according to the nature of the template switched product. If the template switched products comprises or consists of DNA they may for example be amplified by polymerase chain reaction (PCR). If the template switched products comprises or consists of RNA, they may for example be amplified by a reverse-transcription PCR.

Although the methods according to the invention involves the use of at least 2 different templates, however, it is possible to use more than 2 templates. A large number of different starting templates in general allows for the synthesis of a large variety of different template switched products. Hence, if it is preferred to generate a large number of different template switched products, preferably a large number of different templates may be employed.

It is also possible to e.g. shuffle large polynucleotide fragments by providing polynucleotide fragments which are at least pairwise overlapping. Accordingly, it is comprised within the present invention that multiple templates containing partially overlapping sequences can be employed resulting in the formation of a template switched product emerging from multiple template switching events between such templates, wherein said template switched products are longer than the length of each of the individual input templates, but shorter than the sum or combined length of the individual input templates. For example, multiple shorter and partly overlapping templates can be designed and synthesized which each encode only part of a given full-length polynucleotide sequence, and using such a plurality of partly overlapping templates results in the formation of a template switch product. The template switch product results from decoding at least part of each of the templates. Each of the above-mentioned shorter templates can (at least in part) correspond to functional regions or domains of the encoded product, (e.g. a sequence specifying the nature of a chemical entity of a scaffolded molecule, protein structural or functional domains or epitopes), exons of a gene sequence, or the shorter templates can correspond to arbitrarily chosen segments of the full-length polynucleotide sequence.

It should be understood that each of said above-mentioned shorter templates can be a population of homologous templates, for example differing from one another by carrying various nucleotide substitutions, deletions or additions. Such templates carrying substitutions, deletions and/or additions are termed “functional homologues”, “functional equivalents” or “varients” and examples of substitutions, deletions and/or additions are described herein elsewhere in more detail. It will be understood that a variant or functional equivalent can be identified for both the template, or alternatively, for the biological functionality and/or activity encoded by said template or the resulting template switched product generated when exploiting functional homologue templates.

For example, the methods according to the invention may involve the use of more than 2 different templates, such as 3, for example 4, such as 5, for example in the range from 5 to 10, such as from 10 to 20, for example from 20 to 50, such as from 50 to 100, for example from 100 to 200, such as more than 200 different templates.

In some embodiments of the invention, one or more of the templates comprises at least one template switch signal. Said template switch signal may be inserted into the template during synthesis of the template or subsequent to synthesis of the template. Examples of useful template switchs signals are given herein below.

The templates to be employed with the present invention may be synthesised by any suitable method known to the person skilled in the art. The method to be used is dependent on the kind of template.

In one preferred embodiment of the invention the template comprises RNA or an RNA analogue.

RNA may be synthesised in vitro or in vivo. For example RNA templates for reverse transcription may be directly purified from cell extracts or chemically synthesised. Preferably RNA templates are synthesised using in vitro transcription reactions, in which a double-stranded DNA template directs the synthesis of single-stranded RNA. Suitable in vitro transcription reactions are known to the person skilled in the art.

Double-stranded DNA templates to be used in transcription reactions may for example be generated by PCR. Primers used in the PCR reaction are designed to flank the target sequences that are to be used as first templates/second templates or further templates in the methods of the invention. One primer contains a 5′ extension that contains the promoter sequence for a particular RNA polymerase or a functional homologue thereof, e.g. T7 RNA polymerase, such that the promoter will be fused to target sequences in the amplified product. Target sequences may be any DNA and RNA sequences that can be amplified by PCR or RT-PCR. DNA-templates may also be chemically synthesized or be produced by cloning techniques fusing a promoter to target sequences.

Once a suitable double-stranded DNA template is available RNA synthesis may be performed using an in vitro transcription reaction. Double-stranded DNA templates, ribonucleotides and RNA polymerase is mixed in reaction buffer and incubated under conditions allowing for transcription to proceed. The exact conditions depends on the particular RNA polymerase and may for example include incubation at 37° C.

It should be noted that transcription can be carried out using reagents or conditions affecting the fidelity of the process which in some experimental settings could prove useful in generating added diversity to the input sequences. For instance, a T7 RNA polymerase mutant with increased nucleotide substitution error rate has been reported (Brakmann & Grzeszik, 2001, Chembiochem 2, 212-219).

It is also comprised within the present invention that one or more templates are synthesised chemically by conventional method. For example, when the template is an RNA molecule, said RNA molecule may be chemically synthesised. However, in particular when the template is a nucleic acid analogue or comprises nucleotide analogues it is frequently preferred, that such templates are synthesised chemically.

In some embodiments the template may be synthesised in vivo. In particular when the template is a naturally occurring nucleic acid, it is frequently useful that the template is synthesised in vivo. For example the template may be directly purified from a cell extract, i.e. when the template is an RNA or a DNA molecule said RNA or DNA molecule may be purified from a cell extract by any conventional method known to the person skilled in the art.

The cell extract, may be prepared from a number of different cells depending on the specific embodiment, such as but not limited to mammalian cells including human cells, insect cells, plant cells, bacteria, fungi or yeast.

In preferred embodiments of the invention, one or more templates comprises at least one template switch signal. Template switch signals are described in more detail herein below. One kind of template switch signal that may be employed with the present invention is a breakage of the template. If the template is an RNA molecule, said break may for example be inserted directly into the RNA molecule.

In order to generate the maximal diversity breaks may be introduced randomly in a variety of positions, hence generating a number of different DNA or RNA molecules of various length, that each may be transcribed or replicated to generate template. In another embodiment of the invention one or more templates have been prepared by a method comprising the steps of
i) providing a RNA molecule; and
ii) fragmenting said RNA molecule into RNA fragments; and
iii) replicating one or more of said RNA fragments; and
iv) thereby obtaining one or more different templates

Fragmentation may be performed by any method available to the person skilled in the art. For example the DNA or RNA may be fragmented by enzymatic digestion, by limited enzymatic digestion or by limited hydrolysis. The enzymatic digestion may be performed by any suitable nuclease, such as an endonuclease. In case of DNA the endonuclease may for example be a restriction enzyme or DNase I, in case of RNA the endonuclease may for example be an RNase.

Furthermore, it is possible to incorporate phosphorothioate analogues of ribonucleotide during in vitro transcription, which will allow controlled 5′ (prime) to 3′ (prime) exonucleolytic truncation of RNAs as exonuclease stalls at a phoshorothioester.

Polymers and Scaffolded Molecules

A polymer may be any linear or branched molecule consisting of a plurality of monomers. The monomers constituting a polymer may be identical, similar or different. In one embodiment the polymer may for example be a peptide or a polypeptide. If the polymer is a peptide or a polypeptide the monomers are selected from the group consisting of α-amino acids.

In one embodiment of the invention the template switched product encodes polymer comprising or consisting of amino acids. The term “amino acid” should be understood broadly and includes but is not limited to α-amino acids, β-amino acids, γ-amino acids and ω-amino acids. Amino acids may be in any steric form, for example amino acids may be L-amino acids or D-amino acids. In one embodiment of the present invention the polymer consists of or comprises naturally occurring amino acids.

Non-limited examples of polymers includes:
alpha-, beta-, gamma-, and omega-peptides
mono-, di- and tri-substituted peptides
L- and D-form peptides
cyclohexane- and cyclopentane-backbone modified beta-peptides
vinylogous polypeptides
glycopolypeptides
polyamides
vinylogous sulfonamide peptide
Polysulfonamide
conjugated peptide (i.e., having prosthetic groups)
Polyesters
Polysaccharides
Polycarbamates
Polycarbonates
Polyureas
poly-peptidylphosphonates
Azatides
peptoids (oligo N-substituted glycines)
Polyethers
ethoxyformacetal oligomers
poly-thioethers
polyethylene glycols (PEG)
Polyethylenes
Polydisulfides
polyarylene sulfides
Polynucleotides
PNAs
LNAs
Morpholinos
oligo pyrrolinone
polyoximes
Polyimines
Polyethyleneimine
Polyacetates
Polystyrenes
Polyacetylene
Polyvinyl
Lipids
Phospholipids
Glycolipids
polycycles (aliphatic)
polycycles (aromatic)
polyheterocycles
Proteoglycan
Polysiloxanes
Polyisocyanides
Polyisocyanates
Polymethacrylates

Non-limiting examples of monomers constituting a polymer according to the invention includes:
Hydroxyls
Primary, secondary, tertiary amines
Carboxylic acids
Phosphates, phosphonates
Sulfonates, sulfonamides
Amides
Carbamates
Carbonates
Ureas
Alkanes, Alkenes, Alkynes
Anhydrides
Ketones
Aldehydes
Nitatrates, nitrites
Imines
Phenyl and other aromatic groups
Pyridines, pyrimidines, purines, indole, imidazole, and heterocyclic bases
Heterocycles
polycycles
Flavins
Halides
Metals
Chelates
Mechanism based inhibitors
Small molecule catalysts
Dextrins, saccharides
Fluorescein, Rhodamine and other fluorophores
Polyketides, peptides, various polymers
Enzymes and ribozymes and other biological catalysts
Functional groups for post-polymerization/post activation coupling of functional groups
Drugs, e.g., taxol moiety, acyclovir moiety, “natural products”
Supramolecular structures, e.g. nanoclusters
Lipids
Oligonucleotides, oligonucleotide analogs (e.g., PNA, LNA, morpholinos)

In one embodiment of the invention the one or more template switched products encodes a scaffolded molecule. A scaffolded molecule according to the present invention comprises a molecular entity covalently linked to at least 3 functional groups. The functional group may be any molecular entity, the identity of which is encoded by the template or template switched product. For example any of the above mentioned monomers may be a functional group.

Activity of the Templates and the Template Switched Products.

In preferred aspects the present invention relates to methods of providing template switched products comprising a predetermined activity. For example the methods may be used to prepare template switched products with improved activity, using templates comprising said activity.

By way of example, if it is desired to obtain a molecule with improved affinity to a specific compound, one or more templates encoding molecules with affinity for said specific compound may be used. The template switched products may be analysed and products encoding molecules with enhanced affinity for said specific compound may be selected.

Hence, in one preferred embodiment the methods according to the invention comprises that
i) the first template comprises a first activity or encodes a molecule comprising a first activity; and
ii) the second template comprises a second activity or encodes a molecule comprising a second activity; and
iii) the predetermined property of the template switched product is a third activity, either comprised within the template switched product or in a molecule encoded by the template switched product; and
wherein the first activity is different to the third activity and the second activity is different to the third activity.

The activity of the first template, the second template or any other templates or of the recombinded products may individually be for example that the template(s) or product(s) are capable of associating with a specific binding partner with a specific affinity, capable of catalysing a specific reaction, capable of inhibiting a specific reaction, capable of effecting a particular cellular response, capable of interacting with a physical surface or that the template(s) or product(s) encode a molecule capable of associating with a specific binding partner with a specific affinity, capable of catalysing a specific reaction, capable of inhibiting a specific reaction, capable of effecting a particular cellular response or capable of interacting with a physical surface.

Nucleic Acid Polymerase

A nucleic acid polymerase is a catalyst, e.g. an enzyme or ribozyme, capable of catalysing template dependent polymerisation of nucleotides. Preferred nucleic acid polymerases according to the present invention includes such nucleic acid polymerases that are capable of performing one or more template switches during said polymerisation. I.e. preferred nucleic acids polymerases are capable of jumping from one template to another during transcription or replication, thereby synthesising a product which is templated by two or more different templates. Preferred nucleic acid polymerases comprises a RNase H activity.

The nucleic acid polymerase according to the invention may be a naturally occurring nucleic acid polymerase or it may be a nucleic acid polymerase produced by recombinant technology.

In a preferred embodiment the nucleic acid polymerase is a reverse transcriptase, i.e. the nucleic acid polymerase is capable of catalysing polymerisation of DNA using an RNA template. Preferred reverse transcriptases according to the present invention includes but is not limited to HIV reverse transcriptase and functional homologues thereof, reverse transcriptase from MoMLV (moloney murine leukaemia virus) and functional homologues thereof, SNV (spleen necrosis virus) reverse transcriptase and functional homologues thereof or MLV (murine leukaemia virus) reverse transcriptase and functional homologues thereof.

It is also comprised within the present invention to use a nucleic acid polymerase, such as a reverse transcriptase, which has been genetically engineered to enhanced frequency of template switching.

Template Switch Signals

In a preferred embodiment of the invention the methods described herein furthermore comprises introducing into said first and/or second template and/or any further templates one or more template switch signals prior to synthesis of the template switched product. In other embodiments of the invention, one or more templates comprises at least one template switch signal, which was inserted during synthesis of the template(s).

A template switch signal is any modification of a template, capable of inducing a higher rate of template switches of a nucleic acid polymerase

The mechanism of template switch and the factors determining when template switch occurs are still debated (Negroni and Buc, Nat Rev Mol Cell Biol, 2001, 2: 151-155). However, it appears that template switch is promoted when a nucleic acid polymerase for example reverse transciptase encounters sequences or structures at which continued synthesis is difficult (Wu et al., J. Biol. Chem. 1995, 270, 325-332), although evidence for pause independent template switch has also been presented. Below models for template switch are summarised with emphasis on aspects of relevance for the presented invention. However, it will be appreciated by the person skilled in the art that the present invention is not restricted to any of the below described models for template switch.

Pause-driven template switch: Pause-driven template switch occurs when a nucleic acid polymerase encounters obstacles in the template that prevents continued synthesis of product. For example a reverse transcriptase may encounter obstacles in a donor template RNA that prevents DNA synthesis. When this occurs it has been suggested that the RNase H activity of reverse transcriptase degrades the donor template RNA yielding short fragments that dissociate from the nascent DNA strand. Acceptor template RNA can then anneal to the liberated DNA strand and DNA synthesis be resumed. In this model, strand transfer is envisaged as an “enzyme-free” process involving only the nucleic acids.

One natural obstacle to template dependent nucleotide polymerisation catalysed by a nucleic acid polymerase is secondary structure in the template. For example, secondary structures in the template RNA may be an obstacle for reverse transcription. However, the involvement of secondary structures in template switch is not obvious as the secondary structure must be absent in the acceptor template RNA. In natural templates this may be fulfilled if certain secondary structures are formed only after RNase H degradation of 3′ sequences, i.e. the corresponding sequences in non transcribed RNA template does not contain the secondary structure.

A more radical obstacle to continued template dependent nucleotide polymerisation is when a nick or a breakage is present in the template. For example, DNA synthesis by reverse transcriptase will be paused or stopped by a nick or breakage in the template RNA. Reverse transcriptase is then forced to switch template to continue DNA synthesis. This situation termed “forced copy choice” closely resembles strong stop template switch, which occurs during replication of the retroviral genome.

Active displacement: In active displacement, the acceptor template displaces the donor template without requiring strong pausing of nucleic acid polymerase catalysed nucleotide polymerisation. By way of example an acceptor RNA may displace a donor RNA during reverse transcription. The acceptor RNA is thought to hybridize to the newly synthesized DNA strand behind reverse transcriptase. This is possible because the donor RNA is being degraded by the RNase H activity of reverse transcriptase, hence liberating the trailing DNA for interaction with an acceptor RNA. When the acceptor RNA/DNA hybrid catches up, the newly synthesized DNA will anneal to acceptor RNA as soon as the corresponding donor RNA has been degraded. Hence the acceptor RNA is docked onto the nascent DNA behind the RNase H active site and may efficiently compete with the donor RNA for an interaction with reverse transcriptase/the enzyme. However, whether the exchange requires the dissociation of the enzyme from the nascent DNA is currently not known.

A variety of different template switch signals may be employed with the present invention. For example the template switch signal may be a breakage in the template.

In another example the template switch signal comprises a predetermined secondary structure. Said predetermined secondary structure may for example comprise a double stranded nucleic acid, such as a hairpin loop of said nucleic acid. For example the predetermined secondary structure may comprise a double stranded RNA or it may comprise a double stranded DNA, or a double stranded nucleic acid, wherein one of the strands or both of the strands are selected from the group consisting of DNA, RNA, LNA and PNA, including any combination thereof, such as e.g. DNA:DNA; RNA:RNA; DNA:RNA; DNA:LNA; DNA:PNA; RNA:LNA; RNA:PNA.

It is generally known that reverse transcriptase is paused when encountering regions with strong secondary structure. Therefore template switching may be induced with short LNA or PNA oligos hybridized to RNA templates. One advantage of this method is that it is possible to control the sites of recombination and the frequencies of recombination at these sites. Since the frequency of template switching might be expected to correlate with the stability of secondary structures encountered, the frequency of template switching may be controlled by adjusting the length and concentration of LNA/PNA oligos.

In yet another embodiment of the invention the template switch signal comprises a nucleotide analogue. When a template comprises a certain kind of nucleotide analogues, a nucleic acid polymerase may be paused on said template in the position of said nucleotide analogue. According to one theory pausing of the nucleic acid polymerase may cause a template switch, and hence such a nucleotide analogue may serve as a template switch signal.

For example template RNA may contain modified nucleotides that cannot be reverse transcribed. When reverse transcriptase encounters such modified nucleotides, DNA synthesis can only continue if a template switch occurs. Modified nucleotides that may be employed in this approach include abasic nucleotides (sugar phosphate without base), universal nucleotides (non hydrogen bonding) and pyrene nucleotides (base corresponds to entire Watson Crick base pair) (Kunkel and Bebenek, Annu Rev Biochem, 2000, 69: 497-529). Modified nucleotides can be incorporated in the template RNAs either during transcription or as phosphoramidite analogues during chemical synthesis.

In a preferred embodiment of the invention the template switch signal is a breakage in the template. Said breakage may be introduced during synthesis of the template. Methods of introducing these kind of breakages are discussed herein above.

However, breakages may also be introduced into the template subsequent to synthesis of the template. A number of different methods of introducing breakages into nucleic acids or nucleic analogues are known to the person skilled in the art.

For example the method may comprise treatment of the first and/or second template with iodine.

Furthemore, breakages may be introduced by limited enzymatic digestion. Said enzymatic digestion may be catalysed by any suitable enzyme depending on the nature of the tempalte. When the template comprises or consists of RNA, the enzymatic digestion may for example be performed by a ribonuclease.

The breakage(s) may also be introduced by limited alkaline hydrolysis or by limited fragmentation using hydroxyl radicals.

In one embodiment of the invention, the template is an RNA molecule. Breakages may be introduced into RNA by fragmentation of said RNA. Fragmentation of RNA may be carried out in several ways including limited enzymatic digestion with ribonucleases, limited alkaline hydrolysis and limited fragmentation using hydroxyl radicals. In another approach phosphorothioate ribonucleotide analogues are incorporated into the RNA sequences during in vitro transcription. Treatment of RNA molecules containing phosphorothioate ribonucleotide analogues with iodine specifically cleaves phosphorothioester bonds. Hence the degree of fragmentation can be controlled by adjusting the frequency of phosphorothioate ribonucleotide analogues to ribonucleotides.

It is within the scope of the present invention that more than one different kind of template switch signal may be employed with the methods according to the invention. Even within one template, different template switch signals may be inserted.

Template Switching

According to current models for the mechanisms of retroviral recombination, it appears plausible that the frequency of template switching may be increased by increasing the pause frequency of the nucleic acid polymerase. Also increasing hybridization between nascent DNA and acceptor RNA is expected to increase the frequency of recombination.

Hence, depending on the nature of the nucleic acid polymerase, template switching may be induced by a number of other methods apart from introduction of template switch signals. The methods of inducing template switching may be employed alone or in combination with one another and/or in combination with introduction of template switch signals.

Hence in one embodiment of the invention the methods comprises that one or more factors capable of affecting frequency and/or degree and/or accuracy of template switching are added during synthesis of the template switched products.

Said factor can for example be a protein, such as the nucleocapsid protein (NC protein). Furthermore said factor may be a minor groove binding factor or an RNA chaperone, such as StpA.

The conditions under which the synthesis of template switched product is performed may also influence template switching. The processivity and hence pausing frequency of for example reverse transcriptase may be altered by adjusting reaction buffers and in the particular the concentrations of divalent cations and dNTPs. Also the temperature might be changed to decrease processivity. E.g. a temperature shift from 37° C. to 0° C. is expected to pause reverse transcription and influence, such as increase the frequency of template switches.

Hence, in one embodiment of the invention, the synthesis is performed at a temperature in the range of 0 to 5° C., such as 5-15° C., for example 15-25° C., such as 25-50° C., for example 50-70° C.

In another embodiment the synthesis is performed in the presence of a divalent cation at a concentration in the range of from 0.1 mM to about 25 mM, such as from 0.1 mM to 0.5 mM, for example from 0.5 mM to 1 mM, such as from 1 to 2 mM, for example from 2 to 4 mM, such as from 4 to 6 mM, for example from 6 to 7 mM, such as from 7 to 8 mM, for example from 8 to 9 mM, such as from 9 to 10 mM, for example from 10 to 11 mM, such as from 11 to 12 mM, for example from 12 to 14 mM, such as from 14 to 16 mM, for example from 16 to 18 mM, such as from 18 to 20 mM, for example from 20 to 22 mM, such as from 22 to about 25 mM, for example from 5 to 7 mM, such as from 5 to 8 mM, for example from 5 to 9 mM, such as from 5 to 10 mM, for example from 5 to 12 mM, such as from 5 to 14 mM, for example from 5 to 16 mM, such as from 5 to 18 mM, for example from 5 to 20 mM, such as from 5 to 25 mM, for example from 10 to 12 mM, such as from 10 to 14 mM, for example from 10 to 16 mM, such as from 10 to 18 mM, for example from 10 to 20 mM, such as from 10 to 25 mM.

In vivo treatment with hydroxyurea (causing depletion of the cellular dNTP pools and consequently slowing down DNA polymerization by reverse transcriptase) increases template switching (Hwang et al., 2001. Proc. Natl. Acad. Sci., 98, 12209-12214). Thus, in yet another embodiment the synthesis is performed in the presence of dNTPs at a concentration in the range of from 0.1 to 200 μM, for example from 0.1 to 100 μM, such as from 0.5 to 1 μM, for example from 1 to 2 μM, such as from 2 to 4 μM, for example from 4 to 6 μM, such as from 6 to 8 μM, for example from 8 to 10 μM, such as from 10 to 12 μM, for example from 12 to 14 μM, such as from 14 to 16 μM, for example from 16 to 18 μM, such as from 18 to 20 μM, for example from 20 to 25 μM, such as from 25 to 30 μM, for example from 30 to 35 μM, such as from 35 to 40 μM, for example from 40 to 45 μM, such as from 45 to 50 μM, for example from 50 to 55 μM, such as from 55 to 60 μM, for example from 60 to 65 μM, such as from 65 to 70 μM, for example from 70 to 75 μM, such as from 75 to 80 μM, for example from 80 to 85 μM, such as from 85 to 90 μM, for example from 90 to 95 μM, such as from 95 to 100 μM, for example from 1 to 5 μM, such as from 1 to 10 μM, for example from 1 to 15 μM, such as from 1 to 20 μM, for example from 1 to 25 μM, such as from 1 to 30 μM, for example from 1 to 35 μM, such as from 1 to 40 μM, for example from 1 to 45 μM, such as from 1 to 50 μM, for example from 1 to 55 μM, such as from 1 to 60 μM, for example from 1 to 70 μM, such as from 1 to 75 μM, for example from 1 to 80 μM, such as from 1 to 85 μM, for example from 1 to 95 μM.

Furthermore, it is possible to add dNTP analogues that are active site binders, but nonincorporable to the template switched product synthesis reaction, such as a reverse transcription reaction. In this approach the reverse transcriptase is paused by the binding of a dNTP analogue in its active site that cannot be incorporated. Hence the enzyme will pause until the dNTP analogue diffuses out of its active site allowing entrance of a natural (incorporable) dNTP is temporarily blocked. In this approach pausing frequency is controlled by adjusting the ratios of natural dNTP to dNTP analogues. (Wright and Brown, 1985, Biochem Biophys Res Comm., 126, 109-116).

Accordingly, template switching and thereby recombination may be increased by a number of different methods, examples of which are summarised herein below:

A) Fragmentation of RNA
1) Hydrolysis
2) Radical fragmentation
3) Endonucleolytic degradation
4) RNase H degradation mediated by DNA oligos
5) Iodine cleavage of phosphorothioester bonds in RNA incorporated during synthesis (Science Jun. 10, 1988;240(4858):1520-2)

B) Truncation of RNA
1) Dideoxy termination during transcription
2) Fragmentation of DNA template
   i) Radical fragmentation
   ii) Endonucleolytic degradation
   iii) Exonucleolytic degradation
3) Exonucleolytic truncation
   i) Phosphorothioate stopblocks (no refs.)

C) Pausing reverse transcriptase
1) Incorporate modified nucleotide as stop/pause sites (abasic analogues, universal nucleotides, pyrene nucleotides and other nucleotides that cannot be read/reverse transcribed)
2) Hybridize LNA/PNA/morpholino/natural oligos to donor RNA (mimicking secondary structure)
   i) Add oligos and crosslink to donor RNA
3) Add intercalators
4) Modify template with RNA modifying agents (DMS, kethoxal, CMCT, DEPC)

D) Decreasing reverse transcriptase processivity
1) Dope dNTP pool with non-incorporable dNTP mimics
2) Decrease dNTP concentration
3) Add intercalators
4) Decrease divalent cation concentration
5) Low or high temperature

E) Increasing hybridisation between nascent DNA and acceptor RNA
1) Add NC protein (Proc Natl Acad Sci USA Jun. 6, 2000;97(12):6385-90)
2) Add other RNA chaperone, e.g. StpA
3) Add chemicals (e.g. cationic detergents (examples including but not limited to: cetyltrimethylammonium bromide (CTAB), dodecyltri methylammonium bromide (DTAB), tetramethylammonium chloride (TMAC), and the like), modulation of ionic strength by modifying Na(+) and/or K(+) ion concentration, polyethylene glycol (PEG; final concentration of 0.5-20%), dextran sulphate, polyvinylpyrrolidone, and the like)

F) Decreasing hybridisation specificity
1) Increase salt concentration (mono and divalent cations)

G) Affecting strand transfer efficiency of RT
1) Engineering reverse transcriptase for increased efficiency
2) Add (engineered) NC protein
3) Increasing RNaseH activity

H) Addition of RNA oligos (chemically synthesized or transcribed from chemically synthesized DNA templates) to enhance crossover at predetermined positions.

Nucleic Acids and Nucleic Acid Analogues

The following section is directed to nucleic acids, analogues thereof, and templates and/or template switched products comprising such nucleic acids.

Nucleic acids according to the invention comprise a plurality of nucleotides. Nucleic acid analogues comprises one or more nucleotide analogues and optionally one or more nucleotides.

Nucleotides according to the invention includes ribonucleotides comprising a nucleobase selected from the group consisting of adenine (A), uracil (U), guanine (G), and cytosine (C), and deoxyribonucleotide comprising a nucleobase selected from the group consisting of adenine (A), thymine (T), guanine (G), and cytosine (C).

Nucleobases are capable of associating specifically with one or more other nucleobases via hydrogen bonds. Thus it is an important feature of a nucleobase that it can only form stable hydrogen bonds with one or a few other nucleobases, but that it can not form stable hydrogen bonds with most other nucleobases usually including itself. The specific interaction of one nucleobase with another nucleobase is generally termed “base-pairing”.

The base pairing results in a specific hybridisation between predetermined and complementary nucleotides. Complementary nucleotides according to the present invention are nucleotides that comprise nucleobases that are capable of base-pairing.

Of the naturally occurring nucleobases adenine (A) pairs with thymine (T) or uracil (U); and guanine (G) pairs with cytosine (C). Accordingly, e.g. a nucleotide comprising A is complementary to a nucleotide comprising either T or U, and a nucleotide comprising G is complementary to a nucleotide comprising C.

Nucleotide analogues according to the present invention are capable of specific hybridisation and are capable of associating with the active site of at least one nucleic acid polymerase.

Hence, some nucleotide analogues are capable of being incorporated into a growing nucleic acid strand similar to wild type nucleotides, while other nucleotide analogues are capable of being inserted only at the end of a nucleic acid strand and yet other nucleotide analogues are capable of associating with a nucleic acid polymerase, but not capable of being incorporated into a nucleic acid strand.

For example, the nucleotide analogue may be capable of entering the active site of the polymerase, but not capable of being incorporated into a nucleic acid. Furthermore, a nucleotide analogue may be capable of being incorporated to the end of a nucleic acid molecule, but said incorporation of the nucleotide analogue inhibits elongation of the nucleic acid.

In one embodiment of the invention the nucleotide analogue is a phosphorothioate ribonucleotide analogue.

In one embodiment nucleotide analogues include any nucleotide analogues capable of specific base-pairing, for example derivatives of naturally occurring nucleotides or nucleotide analogues wherein the nucleotide backbone differs from naturally occurring nucleotide backbones.

Specific examples of preferred nucleotide analogues useful in this invention include nucleotides analogues containing modified backbones or non-natural internucleoside linkages. As defined in this specification, nucleotide analogues having modified backbones include those that retain a phosphorus atom in the backbone and those that do not have a phosphorus atom in the backbone. For the purposes of this specification, and as sometimes referenced in the art, modified nucleotide analogues that do not have a phosphorus atom in their internucleoside backbone can also be considered to be nucleosides.

Preferred modified nucleotide analogue backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified nucleotide analogue backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above nucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other preferred nucleotide analogues, both the sugar and the internucleoside linkage, i.e., the backbone, of the nucleotide units are replaced with novel groups. The base units are maintained for hybridization with an appropriate nucleic acid target compound. One such nucleotide analogue that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al. (Science, 1991, 254, 1497-1500).

Most preferred embodiments of the invention are nucleotide analogues with phosphorothioate backbones and nucleosides with heteroatom backbones, and in particular —CH₂—NH—O—CH₂—, —CH₂—N(CH₃)—O—CH₂— [known as a methylene(methylimino) or MMI backbone], —CH₂O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are nucleotide analogues having morpholino backbone structures of the above-referenced U.S. Pat. No. 5,034,506.

Modified nucleotide analogues may also contain one or more substituted sugar moieties. Preferred nucleotide analogues comprise one of the following at the 2′ position: OH; F; O—, S—, or N-alkyl, O-alkyl-O-alkyl, O—, S—, or N-alkenyl, or O—, S— or N-alkynyl, wherein the alkyl, alkenyl and alkynyl may be substituted or unsubstituted C₁ to C₁₀ alkyl or C₂ to C₁₀ alkenyl and alkynyl.

Particularly preferred are O[(CH₂)ₙO]ₘCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10.

Other preferred nucleotide analogues comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂, CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 1995, 78, 486-504) i.e., an alkoxyalkoxy group.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the nucleotide, particularly the 3′ position of the sugar on the 3′ terminal nucleotide or in 2′-5′ linked nucleotides and the 5′ position of 5′ terminal nucleotide. Nucleotide analogues may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Nucleotide analogues may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering 1990, pages 858-859, Kroschwitz, J. I., ed. John Wiley & Sons, those disclosed by Englisch et al. (Angewandte Chemie, International Edition 1991, 30, 613-722), and those disclosed by Sanghvi, Y. S., Crooke, S. T. and Lebleu, B., eds., Antisense Research and Applications 1993, CRC Press, Boca Raton, pages 289-302. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2.degree. C. (Sanghvi, Y. S., Crooke, S. T. and Lebleu, B., eds., Antisense Research and Applications 1993, CRC Press, Boca Raton, pages 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. Nos. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941.

Another modification of the nucleotide analogues of the invention involves chemically linking to the nucleotide one or more moieties or conjugates which enhance the activity, cellular distribution or cellular uptake of the nucleotide. Such moieties include but are not limited to lipid moieties such as a cholesterol moiety (Letsinger et al., Proc. Natl. Acad. Sci. USA 1989, 86, 6553-6556), cholic acid (Manoharan et al., Bioorg. Med. Chem. Lett. 1994, 4, 1053-1059), a thioether, e.g., hexyl-S-tritylthiol (Manoharan et al., Ann. N.Y. Acad. Sci. 1992, 660, 306-309; Manoharan et al., Bioorg. Med. Chem. Let. 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J. 1991, 10, 1111-1118; Kabanov et al., FEBS Lett. 1990, 259, 327-330; Svinarchuk et al., Biochimie 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36, 3651-3654; Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett. 1995, 36, 3651-3654), a palmityl moiety (Mishra et al., Biochim. Biophys. Acta 1995, 1264, 229-237), or an octadecylamine or hexylamino-carbonyl-oxycholesterol moiety (Crooke et al., J. Pharmacol. Exp. Ther. 1996, 277, 923-937).

Representative United States patents that teach the preparation of such oligonucleotide analogue conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The present invention also includes nucleic acid analogues which are chimeric nucleic acid analogues. “Chimeric” nucleic acid analogues or “chimeras,” in the context of this invention, are nucleic acid analogues which contain two or more chemically distinct regions, each made up of at least one nucleotide. These nucleic acid analogues typically contain at least one region wherein the nucleic acid analogues is modified so as to confer upon the nucleic acid analogues increased resistance to nuclease degradation, increased cellular uptake, and/or increased binding affinity for the target nucleic acid. An additional region of the nucleic acid analogues may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. This RNAse H-mediated cleavage of the RNA target is distinct from the use of ribozymes to cleave nucleic acids. Ribozymes are not comprehended by the present invention.

Examples of chimeric nucleic acid include but are not limited to “gapmers,” in which three distinct regions are present, normally with a central region flanked by two regions which are chemically equivalent to each other but distinct from the gap. A preferred example of a gapmer is an oligonucleotide in which a central portion (the “gap”) of the oligonucleotide serves as a substrate for RNase H and is preferably composed of 2′-deoxynucleotides, while the flanking portions (the 5′ and 3′ “wings”) are modified to have greater affinity for the target RNA molecule but are unable to support nuclease activity (e.g., fluoro- or 2′-O-methoxyethyl-substituted). Chimeric nucleic acid are not limited to those with modifications on the sugar, but may also include nucleic acid with modified backbones, e.g., with regions of phosphorothioate (P.dbd.S) and phosphodiester (P.dbd.O) backbone linkages or with regions of MMI and P.dbd.S backbone linkages. Other chimeras include “wingmers,” also known in the art as “hemimers,” that is, nucleic acid with two distinct regions. In a preferred example of a wingmer, the 5′ portion of the oligonucleotide serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl-substituted), or vice-versa. In one embodiment, the nucleic acid of the present invention contain a 2′-O-methoxyethyl (2′-O—CH₂ CH₂OCH)₃ modification on the sugar moiety of at least one nucleotide. This modification has been shown to increase both affinity of the nucleic acid analogues for its target and nuclease resistance of the nucleic acid analogues. According to the invention, one, a plurality, or all of the nucleotide and/or nucleotide analogue subunits of the nucleic acid analogues of the invention may bear a 2′-O-methoxyethyl (—O—CH₂CH₂OCH₃) modification. nucleic acid analogues comprising a plurality of nucleotide and/or nucleotide analogue subunits having a 2′-O-methoxyethyl modification can have such a modification on any of the nucleotide analogue subunits within the nucleic acid analogue, and may be chimeric nucleic acids. Aside from or in addition to 2′-O-methoxyethyl modifications, nucleic acid analogues containing other modifications which enhance antisense efficacy, potency or target affinity are also preferred.

The nucleic acids or nucleic acid analogues used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the nucleic acids is well within the talents of the routineer. It is well known to use similar techniques to prepare nucleic acid analogues such as the phosphorothioates and 2′-alkoxy or 2′-alkoxyalkoxy derivatives, including 2′-O-methoxyethyl oligonucleotides (Martin, P., Helv. Chim. Acta 1995, 78, 486-504). It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other conjugated oligonucleotide analogues.

Functional Homoloques

Functional homologues of polypeptides or proteins obtained from template switched products generated according to the present invention comprise any polypeptide or protein sequence which is capable of performing essentially the same function. By way of example, a funtional homologue of HIV reverse transcriptase is capable of essentially performing the function of HIV reverse transcriptase, i.e. catalysing synthesis a DNA molecule using an RNA template.

Functional homologues as e.g. described herein below can be encoded by functional homologue template switched products generated by template switching methods of the present invention employing functional homologues of templates, i.e. templates the incorporation of which into a template switch product results in said template switched product encoding said functional homologue.

Functional homologues according to the present invention comprise polypeptides with an amino acid sequence, which are sharing at least some homology with the predetermined polypeptide sequences as outlined herein above. For example such polypeptides are at least about 40 percent, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with the predetermined polypeptide sequences as outlined herein above.

Homology may preferably be calculated by any suitable algorithm or by computerised implementations of such algorithms for example CLUSTAL in the PC/Gene program by Intelligenetics or GAP, BESTFIT, BLAST, FASTA and TFASTA in the Wisconsin Genetics Software Package, Genetics Computer Group (GCG). The homology between amino acid sequences may furthermore be calculated with the aid of well known matrices such as for example any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Functional homologues may comprise an amino acid sequence that comprises at least one substitution of one amino acid for any other amino acid. For example such a substitution may be a conservative amino acid substitution or it may be a non-conservative substitution.

A conservative amino acid substitution is a substitution of one amino acid within a predetermined group of amino acids for another amino acid within the same group, wherein the amino acids within a predetermined groups exhibit similar or substantially similar characteristics. Within the meaning of the term “conservative amino acid substitution” as applied herein, one amino acid may be substituted for another within groups of amino acids characterised by having
i) polar side chains (Asp, Glu, Lys, Arg, His, Asn, Gin, Ser, Thr, Tyr, and Cys,)
ii) non-polar side chains (Gly, Ala, Val, Leu, Ile, Phe, Trp, Pro, and Met)
iii) aliphatic side chains (Gly, Ala Val, Leu, lie)
iv) cyclic side chains (Phe, Tyr, Trp, His, Pro)
v) aromatic side chains (Phe, Tyr, Trp)
vi) acidic side chains (Asp, Glu)
vii) basic side chains (Lys, Arg, His)
viii) amide side chains (Asn, Gln)
ix) hydroxy side chains (Ser, Thr)
x) sulphor-containing side chains (Cys, Met), and
xi) amino acids being monoamino-dicarboxylic acids or monoamino-monocarboxylic-monoamidocarboxylic acids (Asp, Glu, Asn, Gln).

Functional homologues according to the present invention may comprise more than one such substitution, such as e.g. two amino acid substitutions, for example three or four amino acid substitutions, such as five or six amino acid substitutions, for example seven or eight amino acid substitutions, such as from 10 to 15 amino acid substitutions, for example from 15 to 25 amino acid substitution, such as from 25 to 30 amino acid substitutions, for example from 30 to 40 amino acid substitution, such as from 40 to 50 amino acid substitutions, for example from 50 to 75 amino acid substitution, such as from 75 to 100 amino acid substitutions, for example more than 100 amino acid substitutions.

The addition or deletion of an amino acid may be an addition or deletion of from 2 to 5 amino acids, such as from 5 to 10 amino acids, for example from 10 to 20 amino acids, such as from 20 to 50 amino acids. However, additions or deletions of more than 50 amino acids, such as additions from 50 to 200 amino acids, are also comprised within the present invention.

Additional factors may be taken into consideration when determining functional homologues according to the meaning used herein. For example functional homologues may be capable of associating with antisera which are specific for the poly-peptides according to the present invention.

Peptides with N-terminal alkylations and C-terminal esterifications are also encompassed within the present invention. Functional equivalents also comprise glycosylated and covalent or aggregative conjugates, including dimers or unrelated chemical moieties. Such functional equivalents are prepared by linkage of functionalities to groups which are found in fragment including at any one or both of the N— and C-termini, by means known in the art.

Functional equivalents may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with Met-Leu-Phe. Derivatives of the acyl groups are selected from the group of alkyl-moieties (including C3 to C10 normal alkyl), thereby forming alkanoyl species, and carbocyclic or heterocyclic compounds, thereby forming aroyl species. The reactive groups preferably are difunctional compounds known per se for use in cross-linking proteins to insoluble matrices through reactive side groups.

### EXAMPLES

The following examples illustrates specific embodiments of the invention and should not be regarded as limiting to the invention.

### Example 1

Four polynucleotide sequences are shuffled and subsequently selected on basis of binding to a DNA affinity column.

DNA Oligos

DNA template oligos:
T7pro GACCT CGATC GATCG ATCCT TTAGC CCTAT ATTTA GCTAA TCCGA
ATGGG CAGGA CTTAT ATGGC AGTAG TCATC GCATC-3′
T7pro GACCT CGATC GATCG ATCCT TTAGC CCTAT ATTTA GCTAA CCAGC
GCTTA CAGGA CTTAT ATGGC AGTAG TCATC GCATC-3′
T7pro GACCT CGATC GATCG ATCCT TTAGC CCTAT ATTTA ATCGA TCCGA
GCTTA CAGGA CTTAT ATGGC AGTAG TCATC GCATC-3′
T7pro GACCT CGATC GATCG ATCCT TTAGC CCTAT GTCTG GCTAA TCCGA
GCTTA CAGGA CTTAT ATGGC AGTAG TCATC GCATC-3′
T7pro ≈ T7 RNAP promoter:
5′TAATA CGACT CACTA TAG
Selector oligo:
5′-biotin-GTCTG ATCGA CCAGC ATGGG-3′
DNA primer 1:
5′-TAATA CGACT CACTA TAG GACCT CGATC GATCG ATC-3′
DNA primer 2:
5′-GATGC GATGA CTACT GCC-3′

Preparation of RNA Templates

RNA templates are prepared by T7 RNA polymerase directed in vitro transcription. Double stranded DNA templates for T7 transcription are prepared by PCR with DNA primer 1+DNA primer 2 utilizing the four DNA template oligos as PCR templates. The transcription reaction is performed using standard protocols, e.g. in 40 mM Tris-HCl pH 8.1, 1 mM spermidine, 0.01% Triton X-100, 5 mM Dithiothreitol, 5 mM MgCl₂. 10 mM NaCl, 400 μM each NTP, template DNA and T7 RNA polymerase. The reaction is normally performed at 37° C. After synthesis, DNA templates are digested with RNase free DNase I and synthesized RNA may be either gelpurified, gelfiltrated or phenol-extracted and ethanol precipitated.

Shuffling of Parental RNAs

To enhance template switching the RNA templates from above can be fragmented or other template switch signals may be introduced. Below are described two methods for fragmentation and one method for introducing template switch signals.

A) Fragmentation:
i) Fragmentation by alkaline hydrolysis: RNA samples are added NaOH to 0.5 M and incubated at 50° C. for 1 min, whereafter alkaline hydrolysis is stopped by addition of ½ volumes 1M HCl. After neutralization, samples are gelfiltrated.
ii) Phosphorothioate incorporation and iodoethanol cleavage: Phosphorothioate ribonucleotide analogues are incorporated into RNA templates during transcription. In the described example, a desired frequency of phosphorothioate analogue to natural nucleotides may be 0.05, subsequently giving rise to approximately 1 fragmentation per 20 nt. To achieve this frequency, natural ribonucleotides are used at 1 mM and phosphorthioate analogues at 0.05 mM (Ryder and Strobel, Methods May 1999; 18(1):38-50). After transcription, reactions are treated with RNase free DNase I, phenol-extracted and ethanol-precipitated. Precipitated RNAs are resuspended in water and fragmented by iodoethanol, e.g. by adding a ½ volume of 10 mM Na₂EDTA, 95% formamide, 7% (v/v) iodoethanol followed by heating to 95° C. for 3 minutes (Gish and Eckstein, Science Jun. 10, 1988; 240(4858):1520-2). After fragmentation, formamide and iodoethanol is removed by gelfiltration to allow subsequent reverse transcription of fragmented RNAs

The degree of fragmentation by the various methods is easily analysed by using endlabelled RNA that are resolved on sequencing gels after fragmentation. RNA is endlabeled using T4 polynucleotide kinase and [γ-³²P] ATP using standard protocols.

B) Introduction of template switch signals:

Instead of forcing reverse transcriptase to pause by fragmentation of template RNAs, pausing may be induced by the provoked formation of strong secondary structures in RNA templates. This can be achieved by annealing oligos into template RNAs. Oligos may be composed RNA, DNA, PNA, LNA or other nucleotide analogues. LNA has a very high binding affinity for RNA and may therefore be preferred. The length, sequence and amount of LNA oligomers can be adjusted to suit any particular needs. In this example, LNA oligomers of the length 6 are used at a 1-1 ratio with RNA templates. The sequences of LNA oligos are designed to cover the entire region in which recombination is desired, as illustrated below. LNA oligos are mixed with template RNA and DNA primer-2 in RT-buffer and annealed by slow cooling from 65° C.

```
RNA templates:
        G GACCU CGAUC GAUCG AUCCU UUAGC CCUAU AUUUA GCUAA UCCGA AUGGG CAGGA CUUAU AUGGC
AGUAG UCAUC GCAUC-3′
        G GACCU CGAUC GAUCG AUCCU UUAGC CCUAU AUUUA GCUAA CCAGC GCUUA CAGGA CUUAU AUGGC
AGUAG UCAUC GCAUC-3′
        G GACCU CGAUC GAUCG AUCCU UUAGC CCUAU AUUUA AUCGA UCCGA GCUUA CAGGA CUUAU AUGGC
AGUAG UCAUC GCAUC-3′
        G GACCU CGAUC GAUCG AUCCU UUAGC CCUAU GUCUG GCUAA UCCGA GCUUA CAGGA CUUAU AUGGC
AGUAG UCAUC GCAUC-3′
LNA oligos (3′ to 5′):
                                                TAGGA A
                                                  AGGA AA
                                                    GGA AAT
                                                      GA AATC
                                                        A AATCG
                                                            AATCG G
                                                              ATCG GG
                                                                TCG GGA
                                                                  CG GGAT
                                                                    G GGATA
                                                                        GGATA T
                                                                          GATA TA
                                                                            ATA TAA
                                                                              TA TAAA
                                                                                A TAAAT
                                                                                    TAAAT C
                                                                                      AAAT CG
                                                                                        AAT CGA
                                                                                          AT CGAT
                                                                                            T CGATT
                                                                                                CGATT A
                                                                                                  GATT AG
                                                                                                    ATT AGG
                                                                                                      TT AGGC
                                                                                                        T AGGCT
                                                                                                            AGGCT C
                                                                                                              GGCT CG
                                                                                                                GCT CGA
                                                                                                                  CT CGAA
                                                                                                                    T CGAAT
                                                                                                                        CGAAT G
                                                                                                                          GAAT GT
                                                                                                                            AAT GTC
                                                                                                                              AT GTCC
                                                                                                                                T GTCCT
                                                                                                                                    GTCCT G
                                                                                                                                      TCCT GA
                                                                                                                                        CCT GAA
                                                                                                                                          CT GAAT
                                                                                                                                            T GAATA
                                                                                                                                                GAATA T
                                                                                                                                                  ATTA TA
```

Reverse Transcription

DNA primer-2 (and optionally LNA oligos) is annealed to the template RNAs by mixing and slow cooling from 65° C. in RT buffer (50 mM Tris-HCl, pH 8.0, 75 mM KCl, 0.1 mM EDTA, 1 mM dithiothreitol, and 0.1% Triton X-100), whereafter reverse transcription and hence strand transfer is initated by addition of 100 μM each dNTP, 7 mM MgCl₂ and HIV-1 reverse transcriptase. The reaction is carried out at 37° C.

Separation of cDNAs with a Predetermined Property

After reverse transcription, the recombined cDNA is selected on basis of binding to a selector oligo immobilized via biotin on streptavidin beads (agarose, sepharose, magnetic), or in streptavidin coated tubes.

DNase free RNase I is added to RT reactions to remove RNA templates, whereafter cDNA is gel purified. Streptavidin magnetic beads are prepared by equilibration in binding buffer (e.g. 50 mM K-Hepes pH 7.0, 100 mM KCl) and attachment of the selector oligo via its biotin moiety to streptavidin. cDNAs are then added and the binding reactions are incubated at 25° C. 20 min. Hereafter the binding reactions are washed several times with binding buffer; thus cDNAs are selected on basis of their ability to hybridise to the selector oligo.

The selected sequences may be amplified by PCR using streptavidin magnetic beads with selected cDNAs directly as templates in the PCR. Alternatively, selected cDNAs may be eluted with 200 mM KCl, 100 mM NaOH pH 13. After elution, the pH is adjusted with addition of 1/10 volume 1M HCl. If the selected sequences are captured on the selector oligo immobilized in streptavidin coated PCR tubes, PCR can be performed directly without further handling of the sample.

PCR amplification is carried out with primer-1 and primer-2 using standard procedures. PCR products are then purified and may serve as templates in another round of RNA synthesis, recombination and selection or they may be sequenced for analysis.

1. A method for preparing a template switched product encoded by at least part of one first template and at least part of at least one second template, wherein said product comprises at least one predetermined property, said method comprising the steps of
i) providing a first template molecule and at least one second template molecule; and
ii) providing a nucleic acid polymerase; and
iii) synthesising a plurality of different template switched products by contacting sequentially in any order, or simultaneously, at least part of the first template and at least part of the at least one second template with said polymerase under conditions allowing for template dependent nucleotide polymerisation,
wherein the synthesis of each individual template switched product involves at least one template switch,
and wherein the synthesis of the plurality of different template switched products involves a plurality of template switches,
iv) separating at least one template switched product comprising the at least one predetermined property from said plurality of template switched products; and
v) obtaining a template switched product comprising at least one predetermined property

2. The method of claim 1, wherein
i) the first template comprises a first activity or encodes a molecule comprising a first activity; and
ii) the second template comprises a second activity or encodes a molecule comprising a second activity; and
iii) the predetermined property of the template switched product is a third activity, either comprised within the template switched product or in a molecule encoded by the template switched product; and
wherein the first and second activities are both different from the third activity.

3. (canceled)

4. The method of claim 1, wherein the method involves more than two different template switches.

5-6. (canceled)

7. The method of claim 1, wherein the nucleic acid polymerase comprises a RNase H activity.

8. The method of claim 1, wherein said method involves providing in a single reaction mixture more than 10 different templates and obtaining more than 100 different template switched products.

9. The method of claim 1, wherein the polymerase comprises reverse transcriptase activity.

10-13. (canceled)

14. The method of claim 1, wherein the first template and/or the second template are nucleic acid molecules.

15-21. (canceled)

22. The method of claim 1, wherein the first template and/or the second template are RNA molecules.

23-25. (canceled)

26. The method of claim 1, wherein the method furthermore comprises introducing into said first and/or second template one or more template switch signals prior to synthesis of the template switched product.

27. The method of claim 26, wherein the template switch signal is a breakage in the template.

28. The method of claim 26, wherein the template switch signal comprises a predetermined secondary structure.

29-32. (canceled)

33. The method of claim 26, wherein the template switch signal comprises a nucleotide analogue.

34. The method of claim 33, wherein the nucleotide analogue is capable of entering the active site of the polymerase, but not capable of being incorporated into a nucleic acid.

35. The method of claim 33, wherein the nucleotide analogue is capable of being incorporated to the end of a nucleic acid molecule, and wherein incorporation of the nucleotide analogue inhibits elongation of the nucleic acid.

36. The method of claim 33, wherein the nucleotide analogue is a phosphorothioate ribonucleotide analogue.

37. (canceled)

38. The method of claim 27, wherein the nick(s) are introduced by limited enzymatic digestion.

39. The method of claim 38, wherein the enzymatic digestion is performed by a ribonuclease.

40. The method of claim 27, wherein the breakage is introduced by limited alkaline hydrolysis.

41. The method of claim 27, wherein the breakage is introduced by limited fragmentation using hydroxyl radicals.

42. The method of claim 1, wherein the synthesis comprises addition of one or more factors capable of affecting frequency and/or degree and/or accuracy of template switching.

43. The method of claim 42, wherein said factor is selected from the group consisting of DMS, kethoxal, CMCT, and DEPC.

44-52. (canceled)

53. The method of claim 1, wherein one or more templates have been prepared by a method comprising the steps of
i) Providing a DNA molecule; and
ii) Providing a mixture of nucleotides and nucleotide analogues, wherein one or more of said nucleotide analogues once incorporated into a nucleic acid do not allow further elongation; and
iii) Contacting said DNA molecule with said mixture and transcribing and/or replicating said DNA; and
iv) thereby obtaining one or more different templates.

54. The method of claim 1, wherein one or more templates have been prepared by a method comprising the steps of
i) providing a RNA molecule; and
ii) fragmenting said RNA molecule into RNA fragments; and
iii) replicating one or more of said RNA fragments; and
iv) thereby obtaining one or more different templates

55-56. (canceled)

57. The method of claim 1, wherein the method comprises amplification of the plurality of template switched products.

58. The method of claim 57, wherein said amplification involves a PCR reaction.

59. The method of claim 1, wherein the sequence of steps are repeated at least once.

60. The method of claim 59, wherein template switched product(s) are used as a first template and/or second template when the method is repeated.

61. (canceled)


SEQUENCE LISTING (54 sequences)

SEQ ID NO: 1 - DNA, 80 bp, Artificial Sequence
  Synthetic construct
gacctcgatcgatcgatcctttagccctatatttagctaatccgaatgggcaggacttatatggcagtagtcatcgcatc

SEQ ID NO: 2 - DNA, 35 bp, Artificial Sequence
  Synthetic construct
gcttacaggacttatatggcagtagtcatcgcatc

SEQ ID NO: 3 - DNA, 80 bp, Artificial Sequence
  Synthetic construct
gacctcgatcgatcgatcctttagccctatatttaatcgatccgagcttacaggacttatatggcagtagtcatcgcatc

SEQ ID NO: 4 - DNA, 80 bp, Artificial Sequence
  Synthetic construct
gacctcgatcgatcgatcctttagccctatgtctggctaatccgagcttacaggacttatatggcagtagtcatcgcatc

SEQ ID NO: 5 - DNA, 18 bp, Artificial Sequence
  Synthetic construct
taatacgactcactatag

SEQ ID NO: 6 - DNA, 20 bp, Artificial Sequence
  Synthetic construct
gtctgatcgaccagcatggg

SEQ ID NO: 7 - DNA, 36 bp, Artificial Sequence
  Synthetic construct
taatacgactcactataggacctcgatcgatcgatc

SEQ ID NO: 8 - DNA, 18 bp, Artificial Sequence
  Synthetic construct
gatgcgatgactactgcc

SEQ ID NO: 9 - RNA, 81 nt, Artificial Sequence
  synthetic construct
ggaccucgaucgaucgauccuuuagcccuauauuuagcuaauccgaaugggcaggacuuauauggcaguagucaucgcauc

SEQ ID NO: 10 - RNA, 81 nt, Artificial Sequence
  synthetic construct
ggaccucgaucgaucgauccuuuagcccuauauuuagcuaaccagcgcuuacaggacuuauauggcaguagucaucgcauc

SEQ ID NO: 11 - RNA, 81 nt, Artificial Sequence
  synthetic construct
ggaccucgaucgaucgauccuuuagcccuauauuuaaucgauccgagcuuacaggacuuauauggcaguagucaucgcauc

SEQ ID NO: 12 - RNA, 81 nt, Artificial Sequence
  synthetic construct
ggaccucgaucgaucgauccuuuagcccuaugucuggcuaauccgagcuuacaggacuuauauggcaguagucaucgcauc

SEQ ID NO: 13 - DNA, 6 bp, Artificial Sequence
  Synthetic construct
aaggat

SEQ ID NO: 14 - DNA, 6 bp, Artificial Sequence
  Synthetic construct
aaagga

SEQ ID NO: 15 - DNA, 6 bp, Artificial Sequence
  Synthetic construct
taaagg

SEQ ID NO: 16 - DNA, 6 bp, Artificial Sequence
  Synthetic construct
ctaaag

SEQ ID NO: 17 - DNA, 6 bp, Artificial Sequence
  Synthetic construct
gctaaa

SEQ ID NO: 18 - DNA, 6 bp, Artificial Sequence
  Synthetic construct
ggctaa

SEQ ID NO: 19 - DNA, 6 bp, Artificial Sequence
  Synthetic construct
gggcta

SEQ ID NO: 20 - DNA, 6 bp, Artificial Sequence
  Synthetic construct
agggct

SEQ ID NO: 21 - DNA, 6 bp, Artificial Sequence
  Synthetic construct
tagggc

SEQ ID NO: 22 - DNA, 6 bp, Artificial Sequence
  Synthetic construct
ataggg

SEQ ID NO: 23 - DNA, 6 bp, Artificial Sequence
  Synthetic construct
tatagg

SEQ ID NO: 24 - DNA, 6 bp, Artificial Sequence
  Synthetic construct
atatag

SEQ ID NO: 25 - DNA, 6 bp, Artificial Sequence
  Synthetic construct
aatata

SEQ ID NO: 26 - DNA, 6 bp, Artificial Sequence
  Synthetic construct
aaatat

SEQ ID NO: 27 - DNA, 6 bp, Artificial Sequence
  Synthetic construct
taaata

SEQ ID NO: 28 - DNA, 6 bp, Artificial Sequence
  Synthetic construct
ctaaat

SEQ ID NO: 29 - DNA, 6 bp, Artificial Sequence
  Synthetic construct
gctaaa

SEQ ID NO: 30 - DNA, 6 bp, Artificial Sequence
  Synthetic construct
agctaa

SEQ ID NO: 31 - DNA, 6 bp, Artificial Sequence
  Synthetic construct
tagcta

SEQ ID NO: 32 - DNA, 6 bp, Artificial Sequence
  Synthetic construct
ttagct

SEQ ID NO: 33 - DNA, 6 bp, Artificial Sequence
  Synthetic construct
attagc

SEQ ID NO: 34 - DNA, 6 bp, Artificial Sequence
  Synthetic construct
gattag

SEQ ID NO: 35 - DNA, 6 bp, Artificial Sequence
  Synthetic construct
ggatta

SEQ ID NO: 36 - DNA, 6 bp, Artificial Sequence
  Synthetic construct
cggatt

SEQ ID NO: 37 - DNA, 6 bp, Artificial Sequence
  Synthetic construct
tcggat

SEQ ID NO: 38 - DNA, 6 bp, Artificial Sequence
  Synthetic construct
ctcgga

SEQ ID NO: 39 - DNA, 6 bp, Artificial Sequence
  Synthetic construct
gctcgg

SEQ ID NO: 40 - DNA, 6 bp, Artificial Sequence
  Synthetic construct
agctcg

SEQ ID NO: 41 - DNA, 6 bp, Artificial Sequence
  Synthetic construct
aagctc

SEQ ID NO: 42 - DNA, 6 bp, Artificial Sequence
  Synthetic construct
gtaagc

SEQ ID NO: 43 - DNA, 6 bp, Artificial Sequence
  Synthetic construct
gtaagc

SEQ ID NO: 44 - DNA, 6 bp, Artificial Sequence
  Synthetic construct
tgtaag

SEQ ID NO: 45 - DNA, 6 bp, Artificial Sequence
  Synthetic construct
ctgtaa

SEQ ID NO: 46 - DNA, 6 bp, Artificial Sequence
  Synthetic construct
cctgta

SEQ ID NO: 47 - DNA, 6 bp, Artificial Sequence
  Synthetic construct
tcctgt

SEQ ID NO: 48 - DNA, 6 bp, Artificial Sequence
  Synthetic construct
gtcctg

SEQ ID NO: 49 - DNA, 6 bp, Artificial Sequence
  Synthetic construct
agtcct

SEQ ID NO: 50 - DNA, 6 bp, Artificial Sequence
  Synthetic construct
aagtcc

SEQ ID NO: 51 - DNA, 6 bp, Artificial Sequence
  Synthetic construct
taagtc

SEQ ID NO: 52 - DNA, 6 bp, Artificial Sequence
  Synthetic construct
ataagt

SEQ ID NO: 53 - DNA, 6 bp, Artificial Sequence
  Synthetic construct
tataag

SEQ ID NO: 54 - DNA, 6 bp, Artificial Sequence
  Synthetic construct
atatta